(19) United States
(12) Patent Application Publication
(10) Pub. No.: US 2007/0218148 A1
(43) Pub. Date: Sep. 20, 2007

(54) Essential oils inhibit mold on wood

(75) Inventors: Vina W. Yang, Verona, WI (US); Carol A. Clausen, DeForest, WI (US)

Correspondence Address: JANET I. STOCKHAUSEN;USDA FOREST SERVICE, ONE GIFFORD PINCHOT, MADISON, WI, 53705, US

(73) Assignee: United States as represented by Secretary of Agriculture, Washington, DC (US)

(21) Appl. No.: 11/716,429

(22) Filed: Mar. 9, 2007

Related U.S. Application Data
(60) Provisional application No. 60/782,576, filed on Mar. 15, 2006.

(51) Int. Cl. B32B 21/02; A61K 36/534; A61K 36/23

(52) U.S. Cl. 424/747; 424/778; 428/292.4

(57) ABSTRACT

Methods of treating wood lumber to inhibit growth of mold fungi by surface treating the wood lumber with an essential oil being geranium Egyptian, thyme, dill weed or rosemary. Various surface treatments include dipping, spraying, brushing and vapor exposure.

### PRIORITY INFORMATION

This application claims priority to U.S. Provisional Patent Application Ser. No. 60/782,576 filed Mar. 15, 2006, which is also incorporated herein by reference.

### STATEMENT REGARDING FEDERALLY SPONSORED RESEARCH OR DEVELOPMENT

The U.S. Government has certain rights in the invention disclosed herein.

### FIELD OF THE INVENTION

Methods of manufacture including treatment of untreated wood with essential oils for preventing, inhibiting or controlling growth of mold fungi. Methods of treatment include dip and vapor processes. Essential oils include dill weed, geranium Egyptian, rosemary and thyme.

### BACKGROUND OF THE INVENTION

Moisture management remains the most important critical factor for controlling mold growth on wood and wood products during storage, construction and in service. Potential health risks caused by mold growth in houses and non-residential wooden structures have been a major concern for homeowners, building contractors and insurance companies alike. Law suits claiming health problems caused by indoor mold exposure exceeded 2.8 billion dollars in 2002.

Chemical fungicides commonly used to control the growth of mold on wood are not appropriate for many indoor applications. Natural alternatives that are user friendly and demonstrate low toxicity to humans are desirable for indoor applications. Essential oils are known for their natural, non-toxic components including monoterpenes, diterpenes, and hydrocarbons with various functional groups.

In the early 1990's, it was reported that bioactive plant extracts may be effective against bacteria and fungi. (See Muanza K et al., Antibacterial and antifungal activities of nine medicinal plants from Zaire, Int. J. Pharmacog. 32:337-345 (1994); and Muanza D N et al., Screening for antitumor and anti HIV activities of nine medicinal plants from Zaire, Int. J. Pharmacog. 33:98-106 (1995)). Antimicrobial and antifungal activities of essential oils in food applications, pharmaceutical research and other scientific areas have also been reported. (See Cowan M M, Plant products as antimicrobial agents, Clin. Microbiol. Rev. 12:564-582 (1999); Hammer K A et al., Antimicrobial activity of essential oils and other plant extracts, J. Appl. Microbiol. 86:985-990 (1999); Hoffman B R et al., Screening of antibacterial and antifungal activities of ten medicinal plants from Ghana, Pharmaceutical Biology 42(1):13-17 (2004); Mau J L et al., Antimicrobial effect of extracts from Chinese chive, cinnamon and Corni fructus, J. Agric. Food. Chem. 49:183-188 (2001); Sivropoulou A et al., Antimicrobial activity of mint essential oil, J. Agric. Food Chem. 43:2384-2388 (1995); Adam K et al., Antifungal activities of Origanum vulgare subsp. Mentha spicata, Lavandula angustifolia and Salvia fruiticosa essential oil against human pathogenic fungi, J. Agric. Food Chem. 46:1739-1745 (1998); Deferera D J et al., Analysis of essential oil from some Greek aromatic plants and their fungitoxicity on Penicillium digitatum, J. Agric. Food. Chem. 48:2576-2581 (2000); Moretti et al., In vivo activity of Salvia officinalis oil against Botrytis cinera, J. Essent. Oil Res. 10:157-160 (1998); Muller-Riebau F et al., Chemical composition and fungitoxic properties to phytopathogenic fungi of essential oil of selected aromatic plants growing wild in Turkey, J. Agric. Food. Chem. 43:2262-2266 (1995); Rakotonirainy M S et al., Screening for antifungal activity of essential oils and related compounds to control the biocontamination in libraries and archives storage areas, International Biodeterioration ad Biodegradation 55:141-147 (2005); Scheffer T C et al., Fungistatic vapors for control of mold in packages and equipment, Industrial and Engineering Chemistry 38:619-621 (1946); Sridhar S R et al., Antifungal activity of some essential oils, J. Agric. Food. Chem. 512:7596-7599 (2003); and Wang S-Y et al., Antifungal activities of essential oils and their constituents from indigenous cinnamon (Cinnamomum osmophloeum) leaves against wood decay fungi, Bioresource Technology 96:813-818 (2005)).

### SUMMARY OF THE INVENTION

One aspect of the invention is a method of treating wood or cellulose-containing material to inhibit growth of mold fungi comprising the steps or acts of surface treating the cellulose-containing material with an essential oil being geranium Egyptian, thyme or a combination thereof. In an exemplary embodiment, the surface treatment includes dipping, low pressure spraying, high pressure spraying, brushing, misting, fogging, immersing, injecting, pressure treating and other suitable methods of treating the surface of the cellulose-containing material. In another exemplary embodiment, the surface treatment includes dipping, low pressure spraying, high pressure spraying, brushing, misting, fogging, immersing, injecting, pressure treating and other suitable methods of treating the surface of the cellulose-containing material with geranium Egyptian. In another exemplary embodiment, the surface treatment includes dipping, spraying or brushing the cellulose-containing material with thyme.

Another aspect of the invention is a method of treating wood or cellulose-containing material to inhibit growth of mold fungi comprising the steps or acts of vapor treating the cellulose-containing material with an essential oil being dill weed, rosemary or a combination thereof. In an exemplary embodiment, the vapor treatment is passive vapor treatment, which may also be referred to as fumigating. The vapor treatment may also include other suitable methods of vapor treating the surface of the cellulose-containing material. In another exemplary embodiment, the cellulose-containing material is vapor treated with dill weed. In another exemplary embodiment, the cellulose-containing material is vapor treated with rosemary.

In an exemplary embodiment of any of the inventive methods herein, the cellulose-containing material may be any commercially-available or otherwise suitable materials, including wood (such as southern yellow pine), wood products such as wood lumber, engineered composite such as oriented strandboard (“OSB”) composite, engineered composite, paper coated products such as drywall, or ceiling tile.

In another exemplary embodiment of any of the methods, the mold fungi may be any commonly found mold fungi, such as Trichoderma viride, Aspergillus niger, Penicillium chrysogenum or combinations thereof.

### BRIEF DESCRIPTION OF THE DRAWINGS

FIG. 1 shows a bar graph comparing seven different essential oils against mold fungi growth using the vapor method of treatment on SYP, whereby the analysis included mold resistance of SYP specimens exposed to vapors of seven essential oils (each alone) and challenged with three mold fungi individually in a Petri dish test chamber.

FIG. 2 shows a bar graph comparing seven different essential oils against mold fungi growth using the dip stake method of treatment on southern yellow pine (SYP), whereby the analysis included mold resistance of SYP specimens individually dip-treated with seven essential oils (each alone) and challenged with three mold fungi individually in a Petri dish test chamber.

### DETAILED DESCRIPTION OF EXEMPLARY EMBODIMENTS

Seven essential oils were tested. The essential oils included ajowan, dill weed, geranium Egyptian, lemongrass, rosemary, tea tree and thyme. The oils were obtained from New Directions Aromatics Inc., San Francisco, Calif. All oils were used at full strength unless specified otherwise. Major components and functional groups of the tested oils are found in Edwards V, The Aromatherapy Companion, Published by Storey Brooks, Pownal, Vt., pp. 55-62; and Schnaubelt K, Advanced aromatherapy: The science of essential oil therapy, Published by Healing Arts Press, Rochester, Vt., pp. 9-41, which are hereby incorporated by reference.

Fungal strains. Three types of mold fungi were grown on 2% malt agar (Difco, Becton Dickinson & Co., Sparks, Md.): Aspergillus niger 2.242 (provided by University of Virginia), Penicillium chrysogenum PH02 (from Forest Product Laboratory, Madison, Wis.), and Trichoderma viride ATCC 20476. Aureobasidium pullulans was grown on 2% potato dextrose agar (Difco) for 2 weeks explicitly for inoculation of the soil in the tank test chamber. Spore suspensions of remaining test fungi were prepared by washing the surface of each malt agar plate with 10-15 ml of sterile deionized water (DI) according to ASTM standard D4445-91 (ASTM 1998). In one set of tests, a mixture of 3 mold spore suspensions was transferred to a spray bottle and diluted to 100 ml with DI water to yield 3×10⁷ spores/ml. Spores of individual mold strains were prepared the same as described above for subsequent tests on individual test fungi. The spray bottle was adjusted to deliver 1 ml inoculum per spray.

Test Specimens. Southern yellow pine (SYP) specimens (7×20 mm cross section by 7 cm long), cut from southern pine mill ends obtained from a Mississippi sawmill and stored at 0° C. were used in the petri dish chamber method. Test specimens of kiln-dried SYP, cut into a series of 75×100 mm (12.5 mm thick) samples were used in the tank test chamber method.

Dip stake treatment. Five random replicate specimens were dip-treated for 15 seconds in various essential oils. Vegetable oil served as the control. Specimens were held in a closed container overnight at room temperature according to ASTM test methods D4445-91 and D3273-00 (ASTM 1998; 1986) prior to inoculation with spores of the test fungi. Additionally, thyme and tea tree oil dilutions of 1:2, 1:4 and 1:8 were tested individually and in combination for mold resistance for 22 weeks.

Vapor exposure treatment. Five untreated specimens were held overnight at room temperature in a closed glass Petri dish (1 50×250 mm). A small glass dish (4 cm diameter) containing an individual test oil was set beside the specimens prior to inoculation with spores of the test fungi. Vegetable oil served as the control.

Petri dish test chamber. Each Petri dish test chamber (150×25 mm)(B-D Falcon, Los Angeles, Calif.) contained four layers of blotting paper that was saturated with 30 ml DI water and covered with a polyethylene mesh spacer to elevate specimens. Specimens were sprayed with 1 ml of mixed or individual mold spore inoculum 24 hr post-treatment with essential oil. Petri dish test chambers were sealed in polyethylene bags to prevent drying and incubated at 27° C., 70% RH. Specimens were evaluated for mold growth at 4, 6, 10, 12, 16, 20 and 22 week marks and rated on a scale of 0 to 5: 0 indicating no growth and 5 indicating heavy mold growth. Specimen rating ceased when test oils failed to subsequently inhibit growth of test fungi.

Tank test chamber. A self-contained stainless steel environmental chamber (28×20×26 mm) containing water, soil and hangers for suspending test samples was covered with a pitched roof to prevent condensation from dripping onto specimens. Test chambers were set up in a conditioning room at 30° C. and 70% RH. This set-up, a modification of ASTM D3273-00 (ASTM 1986), is a test method for resistance to mold growth on the surface of Interior Coatings in an Environmental Chamber which did not include an internal heater, electrical fan, or water circulator.

Non-sterile top soil was placed in a tray to a depth of 1 inch above the water level. Soil was inoculated with mold spores from three fungi, Aureobasidium pullulans, Aspergillus niger and Penicillium chrysogenum, two weeks before placing the test specimens in the chamber. Test specimens were vertically suspended across the width of the chamber over inoculated soil.

Specimens individually dip-treated with thyme or geranium Egyptian oils were inoculated with a mixed spore suspension 24 hr post-treatment. For the vapor exposure method, a glass Petri dish containing 5 ml dill weed oil was placed on the soil surface for 24 hours before untreated specimens were introduced.

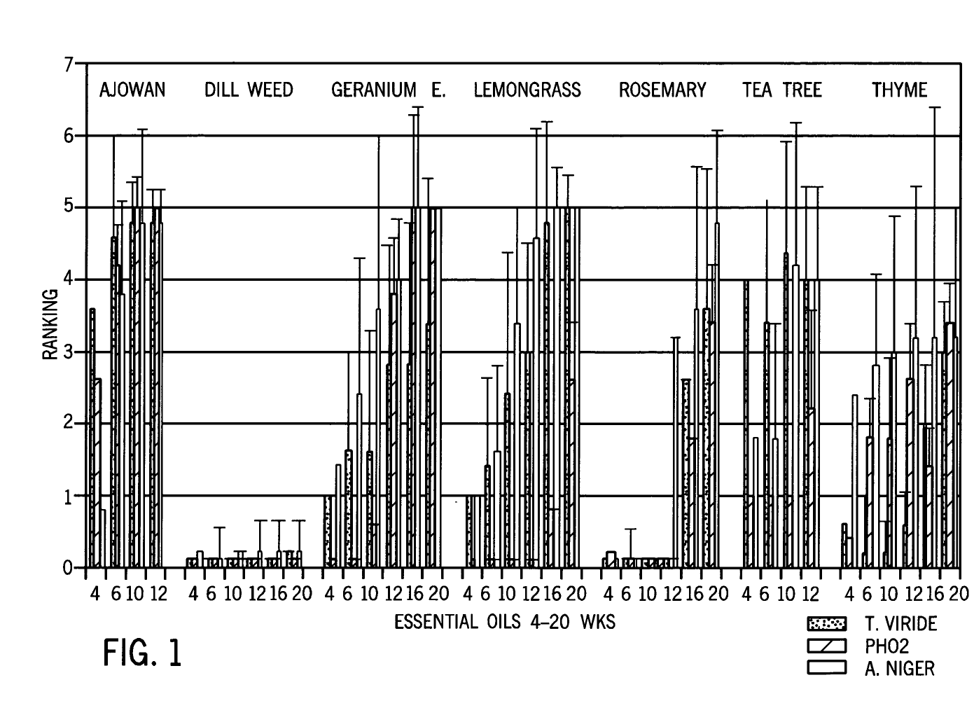

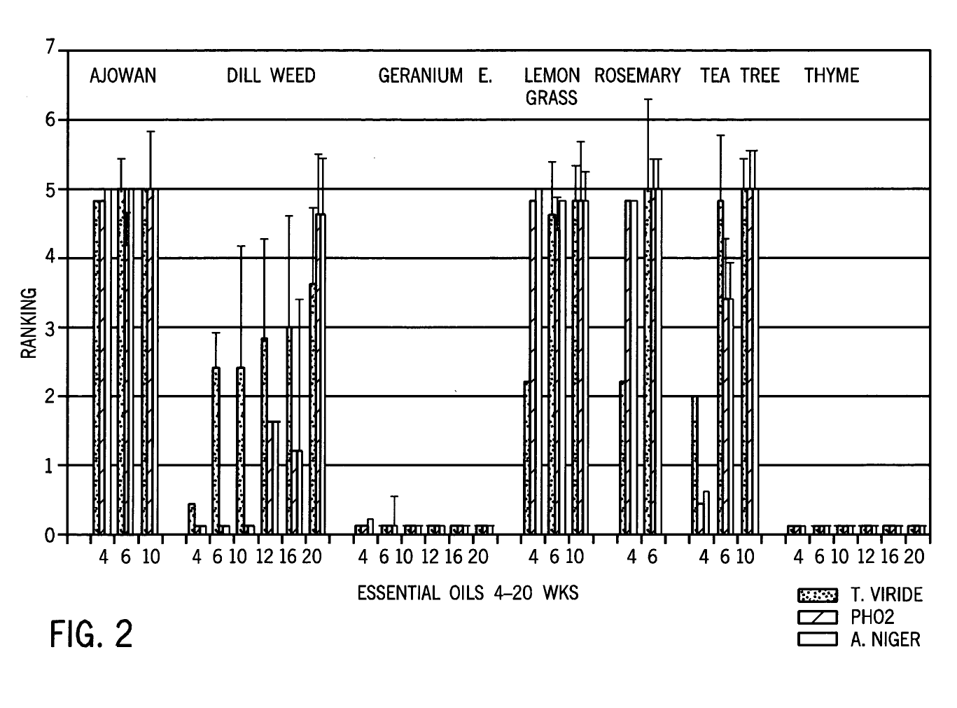

Essential oils were evaluated for antifungal effects on wood against three common air borne mold fungi. The essential oils were assessed using two different methods of treatment: vapor exposure and dip stake. The results are shown in FIGS. 1 and 2, respectively.

Dip stake results. Specimens were initially rated after 4 weeks incubation. Ratings continued periodically through 20 weeks incubation or until test oils failed to substantially inhibit test fungi. Results of the dip stake method showed that ajowan, lemongrass, rosemary and tea tree were about 80% covered with mold growth at week 6 and 100% covered at week 10. The inhibitory effect on the surface of wood specimens was low for ajowan, lemongrass, rosemary and tea tree using the dip stake method of treatment.

In contrast, dill weed oil showed protection against P. chrysogenum PH02 and A. niger, but not against T. viride, for up to 10 weeks. Surprisingly, geranium Egyptian and thyme completely inhibited all test fungi for at least 20 weeks (rated 0 for mold growth). Control stakes dipped in the vegetable oil control showed 100% mold coverage at week 4. Diluted thyme oil (1:8) showed no mold growth up to 22 weeks, while a 1:2 dilution of tea tree oil only demonstrated mold inhibition for 6 weeks. The combination of thyme and tea tree oils was less inhibitory than thyme alone.

Vapor exposure results. Test fungi showed a different response to vapor exposure of essential oils. The most effective mold inhibitor was dill weed vapor. It retarded growth of all three molds for at least 20 weeks. Rosemary vapor inhibited T. viride and Penicillium for 12 weeks and A. niger for 10 weeks (see FIG. 1). These findings may suggest that ketone volatilization likely plays a key role in preventing spore germination for dill weed and rosemary oils. Lemongrass vapor retarded Penicillium growth for 12 weeks, but was ineffective against the other two test mold fungi. Ajowan and tea tree vapors did not inhibit mold fungi. Contrary to dip treatment results, geranium Egyptian and thyme oil vapors did not inhibit mold fungi under the conditions used, which may suggest that the monoterpene components either inhibit spore germination or vegetative growth upon contact.

Petri dish test chamber versus Tank test chamber. Both dip treatment and vapor exposure in the tank test chamber experiment showed positive inhibition for all test fungi on treated specimens for at least 8 weeks. Overall, test results were comparable for the two test apparatuses used.

An important and unexpected observation was that the antifungal properties of thyme and geranium Egyptian oils play an important role in wood protection from mold fungi. The active components of thyme oil (namely geraniol, thymol and carvone) provided significant inhibition of mold growth and serve as a broad spectrum biocide against commonly occurring molds. (See Scheffer T C, 1946). Ajowan was ineffective at inhibiting mold under the conditions used, which is surprisingly contrary to the results reported in Sridhar et al., 2003.

The tendency for high volatilization is advantageous for broadening the range of useful application of essential oils to inhibit mold growth. Vapor inhibition of molds can advantageously provide protection for large volumes of wood products in a closed environment. Dill weed and rosemary oil vapors inhibited mold spores using the vapor exposure method of treatment. Geranium Egyptian and thyme inhibited mold spores using the dip stake method of treatment.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements included within the spirit and scope of the invention, as set forth by the appended claims. The entire disclosures of all references, applications, patents, and publications cited above are hereby incorporated by reference.

We claim:

1. A method of treating a cellulose-containing material to inhibit growth of mold fungi comprising surface treating cellulose-containing material with an essential oil selected from the group consisting of geranium Egyptian, thyme and a combination thereof.

2. The method of claim 1, wherein the surface treating is a member selected from the group comprising dipping, low pressure spraying, high pressure spraying, brushing, misting, fogging, immersing, injecting and pressure treating the cellulose-containing material.

3. The method of claim 2, wherein the essential oil is geranium Egyptian.

4. The method of claim 2, wherein the essential oil is thyme.

5. The method of claim 2, wherein the essential oil comprises a combination of geranium Egyptian and thyme.

6. The method of claim 1, wherein the cellulose-containing material is a member selected from the group comprising wood, wood product, wood lumber, oriented strandboard composite, engineered composite, drywall and ceiling tile.

7. A method of treating cellulose-containing material to inhibit growth of mold fungi comprising vapor treating the cellulose-containing material with an essential oil selected from the group consisting of dill weed, rosemary and a combination thereof.

8. The method of claim 7, comprising passively vapor treating the cellulose-containing material.

9. The method of claim 8, wherein the essential oil is dill weed.

10. The method of claim 8, wherein the essential oil is rosemary.

11. The method of claim 8, wherein the essential oil comprises a combination of dill weed and rosemary.

12. The method of claim 7, wherein the cellulose-containing material is a member selected from the group comprising wood, wood product, wood lumber, oriented strandboard composite, engineered composite, drywall and ceiling tile.
